(19) United States
(12) Patent Application Publication
(10) Pub. No.: US 2017/0198312 A1
(43) Pub. Date: Jul. 13, 2017

(54) NON PURIFIED GLYCEROL

(71) Applicant: PURAC BIOCHEM BV, Gorinchem (NL)

(72) Inventors: Lazar DRASKOVIC, Gorinchem (NL); Wijnand Raphael TERLOUW, Gorinchem (NL); Hendrik Leendert PELT, Gorinchem (NL); David SANCHEZ GARCIA, Gorinchem (NL); Diana VISSER, Gorinchem (NL); André Banier DE HAAN, Gorinchem (NL)

(73) Assignee: PURAC BIOCHEM BV, Gorinchem (NL)

(21) Appl. No.: 15/314,613

(22) PCT Filed: Jun. 5, 2015
(86) PCT No.: PCT/EP2015/062549

§ 371 Date: Nov. 29, 2016

(30) Foreign Application Priority Data
Jun. 6, 2014 (EP) 14171441.0

(51) Int. Cl. C12P 7/52; C12N 1/32; C07C 29/78

(52) U.S. Cl. CPC C12P 7/52; C07C 29/78; C12N 1/32

(57) ABSTRACT

A process including the steps of: providing a glycerol rich-fraction as carbon source to a fermentation medium; fermenting the fermentation medium by means of a microorganism capable of producing propionic acid in the presence of a caustic salt to provide a fermentation broth including a propionic acid salt; and recovering propionic acid salt from the fermentation broth, wherein the glycerol rich-fraction is derived from a process including the steps of: subjecting the glycerol fraction to an evaporative crystallization step to form a distillate fraction including water, and a residue fraction including glycerol and solid salts; and subjecting the residue fraction to a salt removal step, resulting in a salt fraction and a glycerol-rich fraction. The process allows the manufacture of a propionic acid salt using a glycerol-rich carbon source without problems in down-stream processing, and without need for cost-intensive purification steps for the glycerol.

The present invention pertains to a fermentation process using a glycerol-containing material as carbon source, more specifically, a process for manufacturing propionic acid salts via a fermentation process using a glycerol-containing material as carbon source.

Glycerol-based materials are becoming increasingly available, as glycerol is a side product from the manufacture of biodiesel. Biodiesel is a sustainable and renewable fuel produced from various oils and fats. Conventional feedstock for biodiesel manufacture include vegetable and animal lipid materials, more specifically frying and cooking fats, vegetable edible and non-edible oils, such as corn oil, soy oil, palm oil, animal fats from food processing, industrial greases and solvents, and other renewable sources, such as oil from algae and oils and fats produced by fermentation. In the process of manufacturing biodiesel, the oils and fats are decomposed to form fatty acid (esters) and glycerol.

Glycerol has been used as starting material in fermentation processes.

For example, CN101748163 describes a fermentation process for the manufacture of calcium propionate having glycerol as carbon source.

CN102703530 describes a variation on the process of CN101748163 above using a different microorganism.

A. Zhang and S. T. Yang (Process Biochemistry 44 (2009) 1346-1351) also describes a fermentation process for the manufacture of propionate using glycerol as single carbon source.

Yunfen Zhu et al. (Bioresource Technology 101 (2010) 8902-8906) describes the optimization and scale-up of propionic acid production by Propionibacterium acidipropionici with glycerol as the carbon source.

CN101748163 describes also described a fermentation process for the manufacture of propionic acid from glycerol, followed by filtration and spray drying.

A problem occurring in the use of glycerol as starting material in a fermentation process for the manufacturing of propionic acid salts is the following.

Glycerol resulting from biodiesel manufacture, sometimes also indicated as crude glycerol, contains various contaminants including salts and so-called matter organic non-glycerol, also indicated as MONG. Such glycerol can, e.g., comprises 40-80 wt. % of glycerol. 5-10 wt. % of salts, and the balance further components such as methanol, water, and 5-40 wt. % of MONG.

Purified glycerol can, e.g., be obtained by distillation of crude glycerol in combination with a carbon treatment. While purified glycerol is a suitable starting material in a propionate fermentation process, it has the disadvantage that it requires substantial energy input to carry out the necessary distillation steps. On the other hand, it has been found that if crude glycerol is used as starting material in a glycerol-based fermentation, problems occur. These problems are in particular found in downstream processing of the propionate salt, where it appears that product purity is insufficient.

There is therefore need in the art for a method for preparing a glycerol material suitable as carbon source for a propionate fermentation where on the one hand the purification process requires relatively little energy while on the other hand problems in downstream processing are prevented. The present invention provides such a process.

In one embodiment, the invention pertains to a process comprising the steps of
providing a glycerol rich-fraction as carbon source to a fermentation medium;
fermenting the fermentation medium by means of a microorganism capable of producing propionic acid in the presence of a caustic salt to provide a fermentation broth comprising a propionic acid salt, and
recovering propionic acid salt from the fermentation broth, wherein the glycerol rich-fraction is derived from a process comprising the steps of
subjecting the glycerol fraction to an evaporative crystallization step to form a distillate fraction comprising water, and a residue fraction comprising glycerol and solid salts,
subjecting the residue fraction to a salt removal step, resulting in a salt fraction and a glycerol-rich fraction.

In another embodiment the invention pertains to a process comprising the steps of
providing a glycerol fraction comprising glycerol, inorganic salts, and water,
subjecting the glycerol fraction to an evaporative crystallization step to form a distillate fraction comprising water, and a residue fraction comprising glycerol and solid salts,
subjecting the residue fraction to a salt removal step, resulting in a salt fraction and a glycerol-rich fraction,
providing the glycerol rich-fraction as carbon source to a fermentation process wherein a fermentation medium is fermented by means of a microorganism capable of producing propionic acid in the presence of a caustic salt to provide a fermentation broth comprising a propionic acid salt, and
recovering propionic acid salt from the fermentation broth.

In a further embodiment the invention pertains to the use of a glycerol rich-fraction as carbon source in a fermentation process, wherein a fermentation medium is fermented by means of a microorganism capable of producing propionic acid in the presence of a caustic salt to provide a fermentation broth comprising a propionic acid salt, wherein the glycerol rich-fraction is derived from a process comprising the steps of
subjecting the glycerol fraction to an evaporative crystallization step to form a distillate fraction comprising water, and a residue fraction comprising glycerol and solid salts,
subjecting the residue fraction to a salt removal step, resulting in a salt fraction and a glycerol-rich fraction.

It is noted that methods for processing crude glycerol have been described in the art. However, these references describe processes comprise energy intensive processing steps such as distillation, or the glycerol fraction resulting therefrom is disposed of in a different manner, i.e. a manner not involving downstream processing of a carboxylic acid salt, in particular a propionic acid salt.

For example, WO2010/118716 describes a method for the continuous production of pure glycerol from crude glycerol containing potassium sulphate, by the steps of saponifying the organic impurities, evaporating the water, and separating the potassium sulphate by crystallization. The resulting glycerol product is used as combustion material.

DE102007002129 describes the use of disposable grade glycerol as starting material in biogas manufacture.

WO2009/098301 describes a method wherein crude glycerol is subjected to a distillation step to form a pure glycerol phase and a bottom product containing salt and glycerol, and contacting the bottom product containing salt and glycerol with water and an acid. The resulting product can be mixed with further organic material, and used as starting material in an anaerobic fermentation process for manufacture of methane.

E. Nor Hidawati et al. (International Journal of Chemical and Environmental Engineering, October 2011, Volume 2, No. 5, pp. 309-313) describes a process for the treatment of glycerin pitch from biodiesel production which comprises the steps of fatty acid removal by adding water and acidifying to pH 2 followed by liquid-liquid separation, extraction of remaining fatty acid with diethyl ether, evaporation of water at 105° C. to result in a mixture of pure glycerol with inorganic salts, extraction with chilled methanol, salt separation, distillation to remove methanol, and vacuum distillation of the entire product. Many uses of the resulting glycerol are described. Fermentation is mentioned, but fermentation to propionic acid is not. Further, in the purification method of this reference, the entire glycerol product is distilled, and this is not the case for the glycerol product used in the invention.

WO2007/144335 describes subjecting a crude glycerol-based product to at least one treatment, optionally under reduced pressure, of evaporative concentration, evaporative crystallization, distillation, fractional distillation, stripping, or liquid-liquid extraction. The resulting product is used as starting material in the production of dichloropropanol through reaction of glycerol with hydrogen chloride.

EP2486807 describes a process for preparing nutritional, therapeutic, or organoleptic products from crude glycerol by growing yeast under aerobic conditions in a medium containing crude glycerol as carbon source. The resulting yeast product can be processed to obtain nutritional, therapeutic, or organoleptic products such as yeast paste.

WO2013/082309 describes a microorganism suitable for fermenting crude glycerol into organic molecules. No information is provided on downstream processing of the fermentation product.

It has been found that the process according to the invention allows the manufacture of a propionic acid salt by fermentation using a glycerol-rich carbon source without problems in down-stream processing, and without the need for cost-intensive purification steps for the glycerol.

More in particular, the glycerol-rich fraction provided to the fermentation step in the process according to the invention has not been subjected to a glycerol distillation step. This can be seen at least from the fact that the glycerol rich fraction provided to the fermentation step still contains some inorganic salt, in particular at least 0.01 wt. % in total of inorganic salts, more in particular at least 0.05 wt. %, still more in particular at least 0.1 wt. %. Glycerol which has been derived from a distillation step does not contain such amounts of inorganic salt. As the glycerol rich fraction provided to the fermentation step is derived from a salt separation step, it will generally contain less than 5 wt. % in total of inorganic salts, in particular less than 3 wt. %, more in particular less than 1 wt. %.

The propionic acid salt manufactured in the process according to the invention preferably is selected from the group of calcium propionate, magnesium propionate, sodium propionate, and potassium propionate. The propionic acid salt preferably is calcium propionate.

The invention will be elucidated further below.

The starting material in the present invention is a glycerol fraction comprising glycerol, inorganic salt, and water. The glycerol fraction generally has an inorganic salt content of 2-15 wt. %, in particular 5-10 wt. %. The nature of the inorganic salt will depend on the origin of the glycerol-containing fraction. It can, e.g., be one or more of earth alkali metal or alkali metal sulphates, nitrates, or chlorides. It has been found that the process according to the invention is of particular relevancy where the glycerol comprises substantial amounts of sulphate salts, as the presence of these salts has been found to yield fermentation processes where downstream processing shows problems, in particular with regard to contaminant formation in the product. Therefore, in one embodiment, the glycerol fraction has a inorganic sulphate salt content of 2-15 wt. %, in particular 5-10 wt. %.

The glycerol fraction generally comprises water in an amount of 1-30 wt. %, in particular in an amount of 3-15 wt. %.

The glycerol fraction may contain methanol, resulting from the biodiesel manufacturing process, where methanol is used in a transesterification reaction. The methanol content of the glycerol fraction is not critical, and can, e.g., be in the range of 0-10 wt. %, in particular 0-5 wt. %, more in particular 0-3 wt. %.

The glycerol fraction may contain MONG, so-called matter organic non-glycerol. The amount of MONG in a glycerol fraction is defined as any organic matter with is not glycerol or methanol. It is calculated by determining the content of water, methanol, inorganic salts, and glycerol, in a glycerol fraction and subtracting these percentages from 100%. The MONG content of the starting material of the present invention can vary within wide ranges, depending on the source of the glycerol fraction and any pretreatment steps. In one embodiment, the glycerol fraction comprises 0-35 wt. % of MONG. In one embodiment the glycerol fraction can contain 0-10 wt. % of MONG, in particular 0-5 wt. % of MONG. In another embodiment, the glycerol fraction can comprise 5-35 wt. % of MONG, in particular 5-20 wt. % of MONG.

The glycerol content of the glycerol fraction used as starting material in the present invention may vary within wide ranges. It will generally be in the range of 60-95 wt. %, more in particular in the range of 60-90 wt. % glycerol. In one embodiment, the glycerol content in in the range of 60-85 wt. %, more specifically 60-80 wt. %.

The glycerol fraction used in the present invention can be derived from many sources. In one embodiment it is derived from crude glycerol derived from the manufacture of biodiesel.

In one embodiment of the present invention, the glycerol fraction to be subjected to the evaporative crystallization step is obtained from a MONG-removal step, wherein a glycerol fraction comprising 5-35 wt. %, in particular 10-35 wt. % of MONG (and water and salt as described above) is subjected to a MONG removal step to form a glycerol-rich fraction, and a MONG-rich fraction. The glycerol-rich fraction has a MONG content which is less than the MONG content of the glycerol fraction provided to the MONG removal step. The MONG content of the glycerol rich fraction is, e.g., in the range of 0-10 wt. %, in particular 0-5 wt. %.

There are various possibilities for suitable MONG removal steps. In one embodiment, a MONG removal step encompasses allowing the starting material to settle, and then remove the MONG in as far as it has separated out. In a preferred embodiment, a MONG removal step comprises a centrifugation step where the glycerol fraction discussed above is subjected to a centrifugation step to form a glycerol-rich bottom fraction, and a MONG-rich top fraction, and a separation step wherein the MONG-rich top fraction is separated from the glycerol-rich bottom fraction.

In one embodiment water is added to the faction to be provided to the MONG-removal step, where the MONG-removal step is a centrifugation step. The presence of water may result in improved phase separation. A disadvantage is that the water, which will end up in the glycerol-rich fraction will have to be removed during the subsequent evaporative crystallization step. Therefore, if water is added, its amount is preferably limited. In one embodiment water is added to a total water content of the fraction to be provided to the centrifugation step of 1-15 wt. %, in particular 3-10 wt. %.

After the MONG removal step, the glycerol-rich fraction and the MONG rich fraction are separated from each other via a liquid-liquid separation step. Liquid-liquid separation steps are known in the art, and require no further elucidation here.

In the process according to the invention a glycerol fraction comprising glycerol, water, and inorganic salt, and optional further components as described above, is subjected to an evaporative crystallization step. The evaporative crystallization step is carried out under such conditions that water is removed by evaporation, together with methanol, if it is present. The conditions are selected such that glycerol is not evaporated to any material extent. For example, the conditions are selected such that of the water and methanol present, at least 50%, in particular at least 70%, more in particular at least 85%, are removed by evaporation, while of the glycerol and MONG less than 10% is evaporated, in particular less than 5%.

The evaporative crystallisation step can be carried out in manners known in the art. It is within the scope of the skilled person to select suitable evaporative crystallization conditions.

In the evaporative crystallization step, a distillate fraction comprising water is formed, and a residue fraction comprising glycerol and solid salts.

The crux of the evaporative crystallisation step in accordance with the present invention is that the evaporation of water results in a decrease in solubility of the inorganic salts, in particular the sulphate salts, resulting in the formation solid salts in the residue fraction.

The distillate fraction comprises water. If the starting glycerol fraction comprising glycerol, inorganic salts, and water also comprises methanol, the distillate fraction will also comprise methanol.

The amount and composition of the distillate fraction will depend on the amounts of water and methanol present in the feed to the evaporative crystallization step. The top fraction will generally consist for at least 90 wt. % of the total of water and methanol, in particular for at least 95 wt. %, more in particular for at least 98 wt. %.

The residue fraction resulting from the evaporative crystallization step comprises glycerol and solid salts, and may or may not comprise MONG.

The residue fraction is then subjected to a salt removal step, resulting in a salt fraction and a glycerol-rich fraction. The salt removal step in in essence a solid-liquid separation step, where the solid salt is removed from the liquid phase. Suitable solid-liquid separation steps are known in the art, and include, e.g., settling, sedimentation, filtration, centrifugation and the use of apparatus like hydrocyclones. Combinations of various methods may also be used.

Centrifugation may be preferred. It is within the scope of the skilled person to select a suitable method for effecting a solid-liquid separating step.

The resulting glycerol-rich fraction generally comprises less than 5 wt. % of the total of water and methanol, in particular less than 3 wt. %, more in particular less than 2 wt. %. It generally comprises less than 5 wt. % of inorganic salts, in particular less than 3 wt. %, more in particular less than 1 wt. %. The glycerol-rich fraction consists for at least 90 wt. % of the total of glycerol and MONG, in particular at least 95 wt. %, more in particular at least 98 wt. %. The respective amounts of glycerol and MONG in this fraction depend on the amount of MONG present in the starting glycerol. In one embodiment, the amount of glycerol is at least 60 wt. %. It may be preferred for the amount of glycerol to be at least 70 wt. %, more in particular at least 80 wt. %. In some embodiments, where the starting material comprises a relatively low amount of MONG, the glycerol content may be higher, in particular at least 85 wt. %, or at least 90 wt. % of glycerol, or at least 95 wt. % of glycerol.

The salt fraction resulting from the salt separation step generally comprises at least 50 wt. % or inorganic salts, in particular at least 50 wt. % of sodium sulphate and/or potassium sulphate, more in particular for at least 70 wt. %, still more in particular for at least 80 wt. %. Depending on the method for removing the salt, it may be preferred to have some glycerol remaining in the salt fraction, e.g., to form a slurry. In this case, the amount of glycerol may be, e.g., at least 2 wt. %, in particular at least 5 wt. %, e.g., between 2 and 20 wt. %

Where the glycerol-rich fraction resulting from the salt removal step has a MONG content of at least 5 wt. %, e.g., between 5 and 35 wt. %, more in particular between 10 and 35 wt. %, the glycerol-rich fraction can be submitted to a MONG removal step, in particular a centrifugation step, as described above. What has been stated for the MONG removal step there also applies to a MONG removal step centrifugation step that may be carried out on the glycerol-rich fraction resulting from the salt removal step.

The glycerol-rich fraction derived from the salt removal step is, optionally after MONG removal, provided as carbon source in a fermentation medium, which is fermented by means of a microorganism capable of producing propionic acid in the presence of a caustic salt to provide a fermentation broth comprising a propionate salt fermentation product, and recovering the propionate salt from the fermentation broth. It is noted that the glycerol-rich fraction derived from the salt removal step can be provided, optionally after MONG removal, directly to the fermentation step, without further purification steps being required. More specifically, there will be no intermediate glycerol distillation step between the salt removal step and the step of providing the glycerol rich-fraction as carbon source to a fermentation process.

The fermentation can be carried out by methods known in the art, using microorganisms suitable for the production of propionic acid. Examples of suitable microorganisms include Propionibacterium, e.g., P. acidipropionici, P. freudenreichii, P. baumani, and P. thoenii.

It is within the scope of the skilled person to select, using his common general knowledge, a suitable fermentation process, including fermentation conditions, a suitable microorganism, and a suitable broth composition

During the fermentation, the formation of propionic acid results in a decrease in pH of the fermentation broth. To counter this and keep the pH within the range where the microorganism can perform, a caustic salt, generally in the form of a solution is typically added during the fermentation. The addition of the salt results in the conversion of the propionic acid generated to the corresponding propionate salt. Suitable caustic salts include one or more of calcium (hydr)oxide, calcium carbonate, calcium bicarbonate, magnesium (hydr)oxide, sodium hydroxide, ammonium hydroxide, potassium hydroxide, magnesium carbonate, sodium bicarbonate, potassium bicarbonate. Depending on the solubility of the base, the basic solution mentioned above may be a true solution in the sense that the base is completely dissolved and the solution does not contain solid components. However, the basic solution may also be a slurry, which contains solid particles in addition to dissolved base. Within the present specification the word solution is intended to encompass both embodiments. As calcium propionate is a desirable product the addition of a calcium salt as caustic salt is considered preferred. Generally, the basic solution is added in an amount effective to control the pH of the broth between about 3 and 9, more specifically between 6.5 and 8.5.

The fermentation medium will contain other components known in the art such as nitrogen sources, and other constituents.

These do not require further elucidation here.

The glycerol may be used as single carbon source in the fermentation process. It is also possible to possible to combine it with further carbon sources. For the present invention to be attractive, it is generally preferred for the glycerol to make up at least 30 wt. % of the carbon source, preferably at least 50 wt. %, more preferably at least 70 wt. %.

Once the fermentation is completed, the propionic acid salt will be recovered from the fermentation broth.

Generally, the first step in this process is a biomass removal step. This may be carried out in manners known in the art, e.g., via a filtration step or centrifugation step. Efficient biomass removal will improve product quality, including product color.

The resulting product from which biomass has been removed, can be subjected to one or more of the following processing steps:
a purification step, wherein an aqueous stream comprising propionic acid and/or propionic acid salt is purified, e.g., by contacting it with activated carbon, and recovering a purified aqueous stream comprising propionic acid and/or propionic acid salt.
a spray-drying step, wherein an aqueous stream comprising propionic acid and/or propionic acid salt is spray-dried to form a solid powder comprising propionic acid and/or propionic acid salt.
a concentration step, wherein water is removed from an aqueous stream comprising propionic acid and/or propionic acid salt to yield an aqueous stream comprising propionic acid and/or propionic acid salt with a higher concentration.
a precipitation step, wherein contaminants are precipitated from an aqueous stream comprising propionic acid and/or propionic acid salt and precipitatable contaminants, e.g., by adjusting the water content and/or the pH of the medium to such a value that the precipitatable contaminants precipitate from the aqueous medium, while the propionic acid and/or propionic acid salt remain in solution.
a precipitation step, wherein a propionic acid salts is precipitated from an aqueous stream comprising propionic acid and/or propionic acid salt, e.g., by adjusting the water content and/or the pH of the medium to such a value that the propionic acid salt precipitates from the aqueous medium.
an acidification step, wherein an aqueous medium comprising propionic acid salt is acidified by the addition of an acid to convert the propionic acid salt into propionic acid.
an extraction step, wherein an aqueous medium comprising propionic acid is contacted with an organic liquid which is not miscible with water, followed by a phase separation step, wherein the organic liquid comprising propionic acid is separated from an aqueous liquid in which the propionic acid concentration has been reduced.

All steps above are in themselves known in the art. It is within the scope of the skilled person to apply them, separately or in combination, to an aqueous stream comprising propionic acid and/or propionic acid salt. No further elucidation is required.

Preferred processing sequences for recovering propionic acid salt from the fermentation broth are the following:

In a first processing sequence the step of recovering propionic acid salt from the fermentation broth encompasses the sequential steps of biomass removal, optional purification with activated carbon, optionally a concentration step, and spray drying.

In a further processing sequence the step of recovering propionic acid salt from the fermentation broth encompasses the sequential steps of biomass removal, optional purification with activated carbon, a concentration step, an optional precipitation step wherein contaminants are precipitated, and a precipitation step wherein a propionic acid salt is precipitated. This latter step may also be indicated as a crystallization step.

In a further processing sequence the step of recovering propionic acid salt from the fermentation broth encompasses the sequential steps of biomass removal, optional purification with activated carbon, an optional concentration step, an acidification step, and an extraction step.

It has been found that if a glycerol rich fraction prepared as described above is used as carbon source in a glycerol fermentation, the resulting fermentation broth can be processed to relatively pure products. In particular, it has been found that products may be obtained which show less contamination, and/or which show a good stability in that they do not develop undesirable odors, as sometimes occurs when crude glycerol is used as starting material.

It will be clear to the skilled person that preferred embodiments of the various process steps can be combined.

The invention will be elucidated with reference to the following examples, without being limited thereto or thereby.

### EXAMPLE 1

A glycerol purification process was developed using a computer model. The model gave the following results: A starting glycerol fraction was submitted to evaporative crystallization. The crystallization conditions included a temperature of 120° C., and a flash-pressure reduction to 10 mbar. The evaporative crystallization yielded a top fraction comprising water and methanol, and a residue fraction comprising glycerol and solid salts. The residue fraction was centrifuged to form a salt-containing slurry fraction, and a glycerol-rich fraction. The composition of the various fractions is presented in Table 1.

TABLE 1

| | | | | salt- |
|---|---|---|---|---|
| | starting | glycerol | | containing |
| | glycerol | rich | top | slurry |
| Component | fraction | fraction | fraction | fraction |
| glycerol (wt. %) | 75.3 | 85.4 | 0.0 | 8.5 |
| water (wt. %) | 6.2 | 1.1 | 85.0 | 0.1 |
| methanol (wt. %) | 0.9 | 0.0 | 15.0 | 0.0 |
| K2SO4 (wt. %) | 6.0 | 0.4 | 0.0 | 90.0 |
| MONG (wt. %) | 11.5 | 13 | 0 | 1.3 |
| total (wt. %) | 100.0 | 100.0 | 100.0 | 100.0 |

The glycerol-rich product resulting from the evaporative crystallization step can be provided as carbon source to a fermentation process for the production of propionic acid. Optionally, the glycerol-rich fraction can be submitted to a centrifugation step to form a MONG-rich fraction and a glycerol fraction with reduced MONG content which is then provided to the fermentation step.

1. Process comprising the steps of
providing a glycerol rich-fraction as carbon source to a fermentation medium;
fermenting the fermentation medium by means of a microorganism capable of producing propionic acid in the presence of a caustic salt to provide a fermentation broth comprising a propionic acid salt, and
recovering propionic acid salt from the fermentation broth, wherein the glycerol rich-fraction is derived from a process comprising the steps of
subjecting a glycerol fraction comprising glycerol, inorganic salts, and water, to an evaporative crystallization step to form a distillate fraction comprising water and a residue fraction comprising glycerol and solid salts,
subjecting the residue fraction to a salt removal step, resulting in a salt fraction and a glycerol-rich fraction.

2. Process comprising the steps of
providing a glycerol fraction comprising glycerol, inorganic salts, and water,
subjecting the glycerol fraction to an evaporative crystallization step to form a distillate fraction comprising water, and a residue fraction comprising glycerol and solid salts,
subjecting the residue fraction to a salt removal step, resulting in a salt fraction and a glycerol-rich fraction,
providing the glycerol rich-fraction as carbon source to a fermentation process wherein a fermentation medium is fermented by means of a microorganism capable of producing propionic acid in the presence of a caustic salt to provide a fermentation broth comprising a propionic acid salt, and
recovering propionic acid salt from the fermentation broth.

3. Process according to claim 1 wherein the glycerol fraction comprising glycerol, inorganic salt, and water has an inorganic salt content of 2-15 wt. %.

4. Process according to claim 1, wherein the glycerol fraction comprising glycerol, inorganic salt, and water has a water content of 1-30 wt. %.

5. Process according to claim 1, wherein the glycerol fraction to be subjected to the evaporative crystallization step is obtained from a MONG removal step, wherein a glycerol fraction comprising 5-35 wt. %, of MONG is subjected to a MONG removal step to form a glycerol-rich fraction, and a MONG-rich fraction, the MONG removal step comprising a centrifugation step.

6. Process according to claim 5, wherein the MONG content of the glycerol rich fraction is in the range of 0-10 wt. %.

7. Process according to claim 1, wherein the glycerol-rich fraction resulting from the salt removal step comprises less than 5 wt. % of the total of water and methanol.

8. Process according to claim 1, wherein the glycerol-rich fraction resulting from the salt removal step is provided directly to the fermentation step.

9. Process according to claim 1, wherein the glycerol-rich fraction resulting from the salt removal step has a MONG content of at least 5 wt. %, e.g., between 5 and 35 wt. %, and is submitted to a MONG removal step to form a glycerol-rich fraction and a MONG-rich fraction, wherein the glycerol-rich fraction is provided to the fermentation step, wherein the MONG removal step is a centrifugation step.

10. Process according to claim 9, wherein the MONG content of the glycerol rich fraction formed in the MONG removal step is in the range of 0-10 wt. %.

11. Process according to claim 1, wherein the propionic acid salt is selected from the group of calcium propionate, magnesium propionate, potassium propionate, and sodium propionate.

12. Process according to claim 1, wherein the step of recovering propionic acid salt from the fermentation broth encompasses the sequential steps of biomass removal, optional purification with activated carbon, an optional concentration step, and spray drying.

13. Process according to claim 1, wherein the step of recovering propionic acid salt from the fermentation broth encompasses the sequential steps of biomass removal, optional purification with activated carbon, a concentration step, a precipitation step wherein contaminants are precipitated, and a precipitation step wherein a propionic acid salt is precipitated.

14. Process according to claim 1, wherein the step of recovering propionic acid salt from the fermentation broth encompasses the sequential steps of biomass removal, optional purification with activated carbon, an optional concentration step, an acidification step, and an extraction step.

15. A glycerol rich-fraction as carbon source in a fermentation process, wherein a fermentation medium is fermented by means of a microorganism capable of producing propionic acid in the presence of a caustic salt to provide a fermentation broth comprising a propionic acid salt, wherein the glycerol rich-fraction is derived from a process comprising the steps of
subjecting the glycerol fraction to an evaporative crystallization step to form a distillate fraction comprising water and a residue fraction comprising glycerol and solid salts,
subjecting the residue fraction to a salt removal step, resulting in a salt fraction and a glycerol-rich fraction.
